(19) United States
(12) Patent Application Publication
(10) Pub. No.: US 2023/0347103 A1
(43) Pub. Date: Nov. 2, 2023

(54) METHODS AND SYSTEMS FOR PROVIDING SENSORY STIMULATION TO A BRAIN TO TREAT POST-TRAUMATIC STRESS DISORDER

(71) Applicant: Sana Health, Inc., Louisville, CA (US)

(72) Inventor: Richard Hanbury, Lafayette, CO (US)

(73) Assignee: SANA HEALTH, INC., Louisville, CO (US)

(21) Appl. No.: 18/102,587

(22) Filed: Jan. 27, 2023

Related U.S. Application Data
(63) Continuation-in-part of application No. 17/005,047, filed on Aug. 27, 2020, now Pat. No. 11,679,231.
Application No. 17/005,047 is a continuation of application No. 16/422,592, filed on May 24, 2019, now Pat. No. 11,141,559.
Application No. 16/422,592 is a continuation of application No. 15/360,808, filed on Nov. 23, 2016, now Pat. No. 10,328,236.
(60) Provisional application No. 62/258,965, filed on Nov. 23, 2015.

(51) Int. Cl. A61M 21/02; A61M 21/00

(52) U.S. Cl. CPC A61M 21/02; A61M 2021/005; A61M 2209/088; A61M 2205/3592; A61M 2205/3569; A61M 2021/0022; A61M 2021/0027; A61M 2205/505

(57) ABSTRACT

A method of providing sensory stimulation to a user having a neurological disorder, such as Post-Traumatic Stress Disorder, is described. The method includes receiving a set of sensory stimulation instructions by a processor in a headset. The method also includes administering a therapeutically effective amount of a sensory stimulation, wherein the sensory stimulation alternates between a first sensory stimulation including simultaneously providing a left visual stimulus pattern to a left eye of the user and a right auditory stimulus pattern to a right side of the head and a second sensory stimulation including simultaneously providing a right visual stimulus pattern to a right eye of the user and a left auditory stimulus pattern to a left side of the head of the user. The first sensory stimulation and the second sensory stimulation each include a first stimulus pattern, a second stimulus pattern, and a third stimulus pattern.

### CROSS-REFERENCE

This patent application is a continuation-in-part of U.S. application Ser. No. 17/005,047 filed Aug. 27, 2020, titled “Methods and Systems for Providing Stimuli to the Brain,” which is a continuation of U.S. application Ser. No. 16/422,592 filed May 24, 2019, titled “Methods and systems for providing stimuli to the brain” (now published as U.S. Patent Application Publication No. 2019/0321584), which is a continuation of U.S. application Ser. No. 15/360,808 filed Nov. 23, 2016, titled “Methods and Systems for Providing Stimuli to the Brain” (now U.S. Pat. No. 10,328,236). As a continuation of U.S. application Ser. No. 17/005,047, U.S. application Ser. No. 16/422,592, and thus U.S. application Ser. No. 15/360,808, this application claims priority to U.S. Provisional Application No. 62/258,965, filed Nov. 23, 2015, and titled “Methods and Systems for Providing Audio and Visual Stimulus to Treat Neurological Disorders.” All four applications are incorporated by reference herein as if reproduced in full below.

### BACKGROUND OF THE INVENTION

### Field of the Invention

The present disclosure relates to medical devices, systems, and methods. In particular, the present disclosure relates to providing stimuli to a subject to treat various neurological disorders, including Post-Traumatic Stress Disorder (“PTSD”), or conditions and/or to provide performance enhancement.

### Discussion of the Background

Sensory stimulation has been applied to treat various disorders. For example, binaural beats have applied to induce various mental states to encourage sleep, relaxation, meditation, creativity, and other desirable mental states. Combinations of auditory and visual stimuli have been applied to encourage such mental states as well. The application of such therapy, however, has been less than ideal in many circumstances. Equipment to provide the stimulus can be bulky, expensive, generally inaccessible, and below the critical efficacy threshold for widespread use, typically only helping subsets of the population. Users may find the use of such equipment difficult in many circumstances, such as when trying to sleep in a bedroom or an airplane cabin.

To treat various neurological disorders and conditions, pharmaceuticals and/or supplements are often used instead of sensory stimulation. The use of pharmaceuticals, however, can be less than ideal in many circumstances. Often, pharmaceuticals are expensive, rely on patient-compliance, and may require a prescription from a medical professional. Pharmaceuticals may be effective in only a small, less than ideal portion of the general population. To treat insomnia, for example, pharmaceuticals and supplements such as melatonin and zolpidem (e.g., AMBIEN™) have questionable efficacy. Pharmaceuticals often lead to undesirable side effects. For example, some pharmaceutical for treating insomnia can lead to deprivation in certain ranges of deep sleep and increases in mortality rates.

Nonetheless, PTSD is most often treated with psychotherapy or pharmacotherapy. A substantial percentage of patients with PTSD, however, do not recover from the severity and frequency of traumatic flashbacks, nightmares, and/or hypervigilance despite multiple trials of psychotherapy and pharmacotherapy treatments. The lack of recovery and the decrease in patient well-being common when those with PTSD are treated using the standard therapy approaches is a persistent problem in the neurological disorder treatment field. In fact, billions of dollars are spent annually in the United States to supply follow-up pharmacotherapy and psychotherapy treatment and other pill-based treatment approaches to patients with neurological disorders, such as PTSD, that do not respond positively to initial pharmacotherapy and psychotherapy treatment plans.

For at least these reasons, improved methods and systems to treat neurological disorders and other conditions that overcome at least some of the aforementioned challenges are desired.

### BRIEF SUMMARY OF THE INVENTION

The present disclosure relates to medical devices and methods which may be used, for example, to provide stimulus to a subject to treat various neurological disorders or conditions, where the stimulus provided may include one or more of an auditory, a visual, or a tactile stimulus. Examples of neurological disorders which may be treated with devices and methods may include, but are not limited to, insomnia, PTSD, brain injuries including, but not limited to traumatic brain injury (TBI), mild traumatic brain injury (mTBI), or injury from oxygen deprivation of the brain from strokes, depression, anxiety, mood disorders, personality disorders, eating disorders, psychotic disorders, and balance disorders, to name a few. Alternatively or in combination, the stimulus provided by the medical devices and methods described herein may provide cognitive benefits and/or enhancement, including, but not limited to, improving neuroplasticity, motor skills, coordination, reaction times, alertness, energy, working memory, mood, and feelings of wellbeing.

In one aspect, a method of providing sensory stimulation to a user is disclosed. The method includes alternating sensory stimulation between a first sensory stimulation including simultaneously providing a left visual stimulus pattern to a left eye of the user and a right auditory stimulus pattern to the right side of a head of the user and a second sensory stimulation including simultaneously providing a right visual stimulus pattern to a right eye of the user and a left auditory stimulus pattern to the left side of the head. The first sensory stimulation and the second sensory stimulation each include a first stimulus pattern having a first pulse frequency, a second stimulus pattern having a second pulse frequency less than the first pulse frequency, and a third stimulus pattern having a third pulse frequency less than the second pulse frequency. One of the first pulse frequency, the second pulse frequency, or the third pulse frequency is between approximately 3.75 Hz and 4.25 Hz.

In another aspect, an apparatus to provide stimulation to a user is disclosed. The apparatus includes a frame configured to be worn on a head of the user and a controller programmed to generate a plurality of inputs including a left light source input, a right light source input, a left auditory source input, and a right auditory source input. The apparatus further includes a left light source configured to generate a left visual stimulus pattern from the left light source input and a right light source configured to generate a right visual stimulus pattern from the right light source input. The apparatus further includes a left auditory source configured to generate a left auditory stimulus pattern from the left auditory source input and a right auditory source configured to generate a right auditory stimulus pattern from the right auditory source input. The controller is programmed to generate inputs which alternate between a first input including simultaneously generating the left light source input and the right auditory source input and a second input including simultaneously generating the right light source input and the left auditory source input. The first input and the second input each include a first stimulus pattern having a first pulse frequency, a second stimulus pattern having a second pulse frequency less than the first pulse frequency, and a third stimulus pattern having a third pulse frequency less than the second pulse frequency. One of the first pulse frequency, the second pulse frequency, or the third pulse frequency is between 3.75 Hz and 4.25 Hz.

In yet another aspect, a method of treating a neurological disease or condition or providing performance enhancement is disclosed. The method includes providing a headset to be worn by a user and providing sensory stimulation to the user from the headset. The sensory stimulation alternates between a first sensory stimulation including simultaneously providing a left visual stimulus pattern to a left eye of the user and a right auditory stimulus pattern to the right side of a head of the user and a second sensory stimulation including simultaneously providing a right visual stimulus pattern to a right eye of the user and a left auditory stimulus pattern to the left side of the head. The first sensory stimulation and the second sensory stimulation each include a first stimulus pattern having a first pulse frequency, a second stimulus pattern having a second pulse frequency less than the first pulse frequency, and a third stimulus pattern having a third pulse frequency less than the second pulse frequency. One of the first pulse frequency, the second pulse frequency, or the third pulse frequency is between 3.75 Hz and 4.25 Hz.

In another aspect, a method of treating a user having PTSD is disclosed. The method includes receiving a set of sensory stimulation instructions by a processor in a headset configured for being worn on a head of the user. The method further includes, responsive to receiving the set of sensory stimulation instructions, administering a therapeutically effective amount of a sensory stimulation to the user. The sensory stimulation alternates between a first sensory stimulation including simultaneously providing a left visual stimulus pattern to a left eye of the user and a right auditory stimulus pattern to a right side of the head of the user, and a second sensory stimulation including simultaneously providing a right visual stimulus pattern to a right eye of the user and a left auditory stimulus pattern to a left side of the head of the user. The first sensory stimulation and the second sensory stimulation include a first stimulus pattern having a first pulse frequency, a second stimulus pattern having a second pulse frequency, and a third stimulus pattern having a third pulse frequency. The method further includes collecting a set of user data associated with the administration of the therapeutically effective amount of the sensory stimulation to the user.

In another aspect, a headset for providing a therapeutically effective amount of a sensory stimulation to a user is disclosed. The headset includes a frame configured to be worn on a head of the user. The headset also includes a processor configured to generate a plurality of inputs including a left light source input, a right light source input, a left auditory source input, and a right auditory source input. The headset also includes a left light source configured to produce, using the left light source input, a left visual stimulus pattern. The headset also includes a right light source configured to produce, using the right light source input, a right visual stimulus pattern. The headset also includes a left auditory source configured to produce, using the left auditory source input, a left auditory stimulus pattern. The headset also includes a right auditory source configured to produce, using the right auditory source input, a right auditory stimulus pattern. The processor is configured to administer, by the headset, a therapeutically effective amount of the sensory stimulation to the user. The plurality of inputs alternate between a first input including simultaneously generating the left light source input and the right auditory source input, and a second input including simultaneously generating the right light source input and the left auditory source input. The first input and the second input include a first stimulus pattern having a first pulse frequency, a second stimulus pattern having a second pulse frequency less than the first pulse frequency, and a third stimulus pattern having a third pulse frequency less than the second pulse frequency.

In another aspect, system for treating a user having PTSD is disclosed. The system includes a headset configured to be worn on a head of a user. The headset includes a frame, a left light source, a left auditory source, a right light source, and a right auditory source. The system also includes a processor communicatively coupled to the headset and a controller. The processor is configured to receive a sensory stimulation from the controller and transmit the sensory stimulation to the headset. The sensory stimulation is transmitted at a therapeutically effective amount and the sensory stimulation alternates between: a first sensory stimulation including simultaneously providing a left visual stimulus pattern from the left light source to a left eye of the user and a right auditory stimulus pattern from the right auditory source to a right side of the head of the user, and a second sensory stimulation including simultaneously providing a right visual stimulus pattern from the right light source to a right eye of the user and a left auditory stimulus pattern from the right auditory source to a left side of the head of the user. The first sensory stimulation and the second sensory stimulation include a first stimulus pattern having a first pulse frequency, a second stimulus pattern having a second pulse frequency less than the first pulse frequency, and a third stimulus pattern having a third pulse frequency less than the second pulse frequency.

These features together with the various ancillary provisions and features which will become apparent to those skilled in the art from the following detailed description, are attained by the methods and system for providing stimulation to a user of the present invention, embodiments thereof being shown with reference to the accompanying drawings, by way of example only, wherein:

### BRIEF DESCRIPTION OF THE SEVERAL VIEWS OF THE DRAWING

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIGS. 1A and 1B generally illustrate schematic diagrams of therapeutic systems to provide therapeutic auditory, visual, and/or tactile stimulus, according to the principles of this present disclosure;

FIGS. 2A and 2B generally illustrate schematic diagrams of the controller for the therapeutic systems of FIGS. 1A and 1B according to the principles of this present disclosure;

FIG. 3A generally illustrates an exemplary therapeutic wearable headset or sleep mask, according to many embodiments according to the principles of this present disclosure;

FIG. 3B generally illustrates a user wearing the therapeutic wearable headset and sleep mask of FIG. 3A according to the principles of this present disclosure;

FIG. 4 generally illustrates a flow chart of a therapeutic method of providing therapeutic auditory, visual, and/or tactile stimulus, according to several embodiments according to the principles of this present disclosure; and

FIG. 5 generally illustrates a flow chart of a therapeutic method of providing stimuli to the brain of a user with a neurological disorder or condition according to the principles of this present disclosure.

### DETAILED DESCRIPTION OF THE INVENTION

The following discussion is directed to various embodiments of the present disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

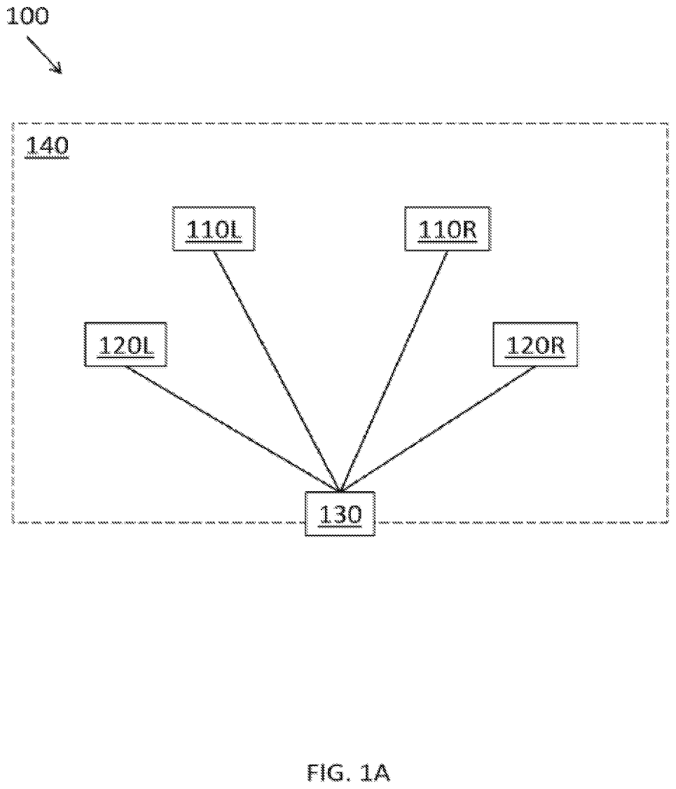

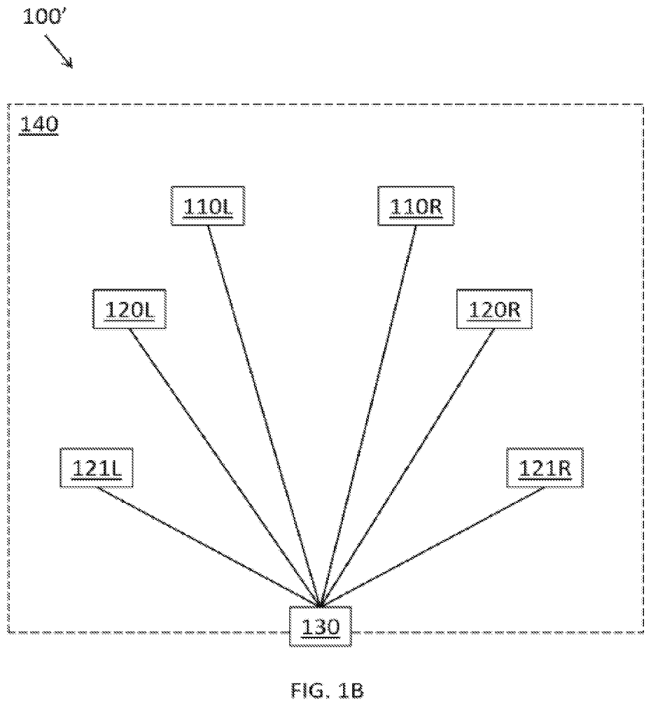

FIG. 1A is a schematic diagram of a first embodiment therapeutic system 100. Therapeutic system 100 provides one or more outputs that a person wearing the therapeutic system may experience as auditory, visual, and/or tactile stimulus. Thus, in one embodiment, therapeutic system may comprise a left light source 110L, a right light source 110R, a left vibration source 120L, a right vibration source 120R, and a controller 130 for independently controlling and coordinating the action of the light and vibration sources. Thus, for example, therapeutic system 100 may be positioned on the head of a user with left light source 110L positioned over the left eye to provide a left visual stimuli, right light source 110R positioned over the right eye to provide a right visual stimuli, left vibration source 120L positioned to provide left ear auditory stimuli, and right vibration source 120R positioned to provide right ear auditory stimuli.

In one embodiment, left and right light sources 110L, 110R may each comprise light-emitting diodes, an incandescent light source having a wavelength filter, a fluorescent light source, a backlit LCD panel, or other light source configured to provide to the user light at a desired, predetermined wavelength or wavelength range.

In another embodiment, left and right vibration sources 120L, 120R may each comprise earbuds, miniature speakers, or other vibration sources that can provide auditory stimuli to a user. In certain other embodiments, left and right vibration sources 120L, 120R may comprise bone conduction transducers in the audible frequency range to provide vibrations to the user's skull bone that is sensed as auditory by the user's ear. Optionally, one or more of left and right vibration sources 120L, 120R may also produce vibrations that are sensed as tactile stimuli. Thus, for example, controller 130 may provide first signals to bone conduction transducers that vibrate or oscillate at a first frequency that can be interpreted by the user as auditory stimuli and may provide second signals at a second, lower frequency that can be interpreted as a tactile sensation by the user. In other words, bone conduction transducers may be adapted to provide both auditory and tactile stimulus to the user.

In certain embodiments, left and right vibration sources 120L, 120R provide output at specific one or more frequencies or a range of frequencies, and are turned on and off at a stimulation frequency. Thus, for example, a vibration source may be programmed to provide an output at an audio frequency of 256 Hz for some period of time, followed by no output for the following period of time. Thus, the vibration source is the product of an audio frequency and a square wave.

FIG. 1B is a schematic diagram of a second embodiment therapeutic system 100′. Second embodiment therapeutic system 100′ is generally similar to first embodiment therapeutic system 100′, except as explicitly noted. Specifically, second embodiment therapeutic system 100′ includes a left tactile stimulus source 121L and a right tactile stimulus source 121R, each of which may be individually controlled and coordinated with the controller 130 to provide tactile stimuli to a user of therapeutic system 100′.

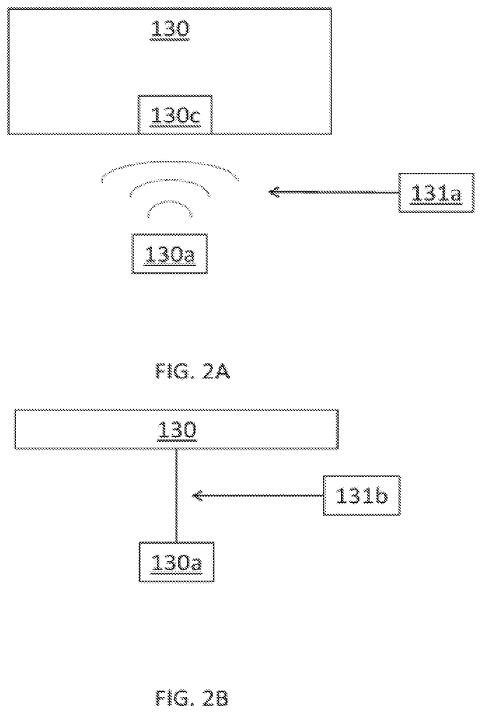

FIGS. 2A and 2B show schematic diagrams of the controller 130 of therapeutic system 100 or 100′. As shown in FIG. 2A, therapeutic system 100 or 100′ may optionally include an external control unit 130a that may wirelessly communicate with a wireless receiver/transmitter 130c of the controller 130 through a wireless connection 131a. The wireless connection 131a may comprise a Bluetooth connection, a Bluetooth LE connection, a WiFi connection, a ZigBee connection, an infrared (IR) connection, a radiofrequency (RF) connection, or an inaudible auditory signal connection, to name a few examples. The external control unit 130a may comprise a custom-built, electronic controller. In many embodiments, the external control unit 130a may comprise a personal computing device of the user that may have downloaded onto and operating, a custom computer application or “app” to operate the system 100 or 100′ to provide a therapeutic regimen. For example, the personal computing device may comprise a personal computer, a personal laptop computer, a tablet computer, or a smartphone. The custom computer application or “app” may be an application or “app” downloadable from an application distribution platform. The application may include one or more therapeutic regimens that the user may select for implementation by the therapeutic system 100 or 100′. In some embodiments, the application may allow the user to provide feedback information about the efficacy of the therapeutic regimen(s), the feedback may be uploaded and collected by a central server(s) in communication with the application, and the therapeutic regimen(s) may be improved or optimized based on the feedback from the one or more users. Alternatively or in combination, as shown in FIG. 2B, the system 100 or 100′ may further comprise an external control unit 130a, such as a custom-built controller, that may communicate with the controller 130 through a wired connection 131a, for example, a USB, FireWire, or Lightning connection, to name a few examples.

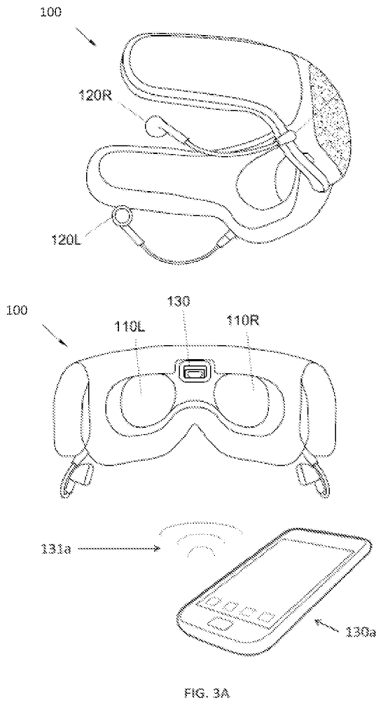

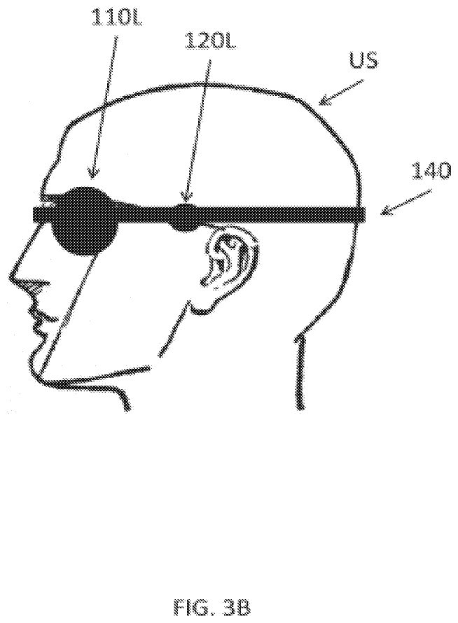

FIG. 3A shows one embodiment of the therapeutic system 100 as including therapeutic wearable headset or sleep mask 140 which integrates the light, vibration, and, optionally, tactile sources into a single form factor for presentation to a user. Thus, for example, when a user places wearable headset or sleep mask 140 on their head, left light source 110L is positioned over the left eye to provide a left visual stimuli, right light source 110R is positioned over the right eye to provide a right visual stimuli, left vibration source 120L is positioned to provide left ear auditory stimuli, and right vibration source 120R is positioned to provide right ear auditory stimuli.

As discussed above and herein, the left vibration source 120L and the right vibration source 120R may each comprise bone conduction transducer that may provide both auditory and tactile stimulus. Alternatively, wearable headset or sleep mask 140 is therapeutic system 100′ which includes left tactile stimulus source 121L and right tactile stimulus source 121R, each of which may be individually controlled and coordinated with the controller 130, as described above regarding FIG. 1B.

As discussed above and herein, the therapeutic wearable headset or sleep mask 140 may be operated with an external controller 130a (e.g., a smartphone) in communication with the controller 130 through a wireless connection 131a, for example. The user US may have an option to turn tactile stimulation on or off, for example. FIG. 3B shows a user US wearing the therapeutic wearable headset or sleep mask 140.

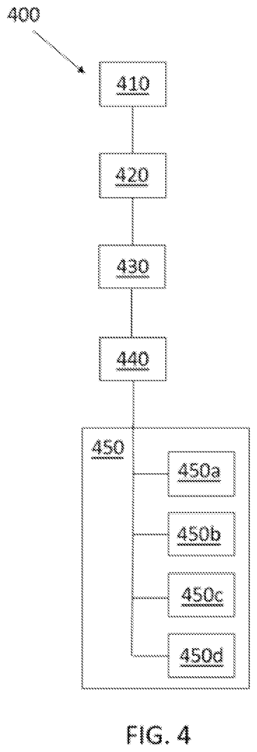

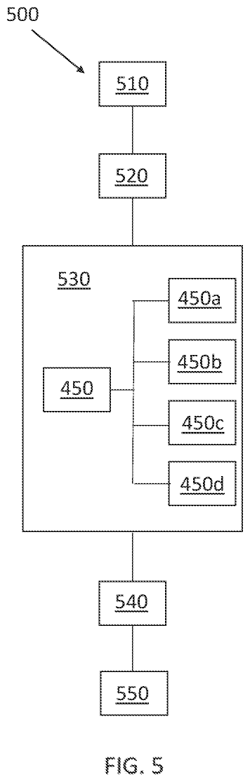

FIG. 4 shows a flow chart of an exemplary therapeutic method 400 for providing therapeutic auditory, visual, and/or tactile stimulus. In a step 410, a subject having a neurological disorder or condition may be identified. Examples of neurological disorders may include, but are not limited to, insomnia, PTSD, brain injuries such as traumatic brain injury (TBI), mild traumatic brain injury (mTBI), or injuries to the brain due to oxygen deprivation, such as strokes, depression, anxiety, mood disorders, personality disorders, eating disorders, and psychotic disorders. Alternatively, a subject may be selected to undergo a therapeutic method 400 for the purpose of performance enhancement of mental and/or physical tasks for to aid the subject in napping or sleeping. In a step 420, the subject may be provided the therapeutic system or headwear, such as the system 100 or 100′ described above. In a step 430, the subject may wear the therapeutic system or headwear, such as wearable headset or sleep mask 140. In a step 440, headset 140 executes programming 450 provided in controller 130 to provide stimuli to the subject. The programming provides two or more of auditory, video, and/or tactile stimulus are concurrently provided by headset 140 to the subject, and thus, for example, may provide power to activate left light source 110L, right light source 110R, left vibration source 120L and or right vibration source 120R.

As discussed above and herein, the left vibration source 120L and the right vibration source 120R may each comprise bone conduction transducer that may provide both auditory and tactile stimulus. Alternatively, wearable headset or sleep mask 140 is therapeutic system 100′ which includes left tactile stimulus source 121L and right tactile stimulus source 121R, each of which may be individually controlled and coordinated with the controller 130, as described above regarding FIG. 1B.

In certain embodiments, providing two or more of auditory, video, and/or tactile stimulus concurrently may provide improved therapeutic benefits as compared to providing only one of auditory, video, or tactile stimulus at one time. The two or more auditory, video, and/or tactile stimulus may thus combine to provide the improved therapeutic benefits, for example (i.e., the two or more auditory, video, and/or tactile stimulus may synergize in a way to provide improved results over providing two of the stimuli individually.)

Exemplary instructions for providing stimuli may be provided, for example, by programming 450, such as a subroutine 450a, which includes the simultaneous activation of all active auditory, video, and/or tactile stimulus sources. Optionally, the activation of all sources may include the activation of tactile stimulation to run throughout all subsequent auditory and/or visual stimulation. Another exemplary subroutine 450b may comprise alternating the left auditory, video, and/or tactile stimulus sources with the right auditory, video, and/or tactile stimulus sources (i.e., the left stimuli and right stimuli take turns being active.) Another exemplary subroutine 450c may comprise alternating the visual sources with the auditory and/or tactile sources (i.e., the visual stimuli and the auditory/tactile stimuli take turns being active.) Another exemplary subroutine 450d may comprise alternating the left auditory and/or tactile source and the right visual source with the right auditory and/or tactile source and the left visual source (i.e., opposite auditory/tactile stimuli take turns being active.) Such programming is further described below.

In step 440, programming 450, including by not limited to subroutines 450a, 450b, 450c, and 450d, may each be applied one or more times, individually or in combination with one another. The programming may, in addition, provide sequences of output in subroutines 450a, 450b, 450c, and 450d at different frequencies and/or timings. Thus for example the subroutines may provide output at specific frequencies that change as the subroutine is repeated. Thus for example, subroutine 450a may provide auditory output to vibration source 120R or 120L at a frequency of 256 Hz that is turned on and off, that is it is pulsed, at a pulse frequency of 1 Hz for 2 minutes. This square pulse auditory signal thus generates signals at a frequency of 1 Hz in addition to higher harmonics. At a subsequent time the output at 256 Hz is pulsed at twice the previous pulse frequency for 2 minutes. In this manner, the auditory frequency of 256 Hz may be modulated over a wide range, including frequencies corresponding to brain wave frequencies.

In addition, by alerting the output between left and right channels, the brain may be stimulated in a way that it is forced to communicate between the left and right sides of the brain. This forced communication, for example, can allow PTSD memories to be wired to both sides of the brain, thereby stopping undesirable flashbacks.

Although the above steps show method 400 of treating a patient in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as beneficial to the treatment.

One or more of the steps of the method 400 may be performed with the circuitry as described herein, for example, circuitry of the controller 130 or the external control unit 130a such as one or more of a processor or logic circuitry such as a central processing unit (CPU) or a programmable array logic for field programmable gate array. The circuitry may be programmed to provide one or more of the steps of the method 400, and the program may comprise program instructions stored on a computer readable memory or programmed steps of the logic circuitry such as the programmable array logic or the field programmable gate array, for example.

FIG. 5 shows a flow chart of an exemplary therapeutic method 500 for providing therapeutic auditory, visual, and/or tactile stimulus to the brain of a user with a neurological disorder or condition. An auditory stimulus can include a tone, beep, song, or any other desired form of sound presented audibly to the user. A visual stimulus can include a flash of white light, a presentation of various colors, display of a visual image, or any other desired form of light presented visually to the user. A tactile stimulus can include a vibration, a tap, a movement of air, or any other desired form of physical touch present tactilely to the user.

In a step 510, a headset 140 can be provided to a user. The user can be a person with a diagnosed or suspected neurological disorder or condition. An example of a neurological disorder may include, but is not limited to, PTSD.

The headset 140 provided in step 510 can be configured to provide an auditory, visual, and/or tactile stimuli to the brain of the user. For example, the headset 140 can include a frame, a processor, a left light source 110L, a right light source 110R, a left auditory or vibration source 120L, and a right auditory or vibration source 120R.

In some embodiments, the headset 140 can include a left tactile stimulus source 121L and right tactile stimulus source 121R, each of which may be individually controlled and coordinated with the controller 130, as described above regarding FIG. 1B.

The processor, using provided programming 450 (discussed in further detail below), can generate a left light source input, a right light source input, a left auditory source input, and/or a right auditory source input. The generated inputs are sent from the processor to the controller 130, which is operatively coupled to the hardware of the headset 140 so that a corresponding output arises from the left light source 110L, the right light source 110R, the left auditory source 120L, the right auditory source 120R, or a combination thereof.

For example, if the processor contains instructions for providing a visual stimuli to the left eye of the user, then the processor can generate a left light source input. In some instances, the generation of a left light source input results in an output of light from the left light source 110L. Accordingly, as another example, if the processor contains instructions for providing a visual stimuli to the right eye of the user, then the processor can generate a right light source input. In some instances, the generation of a right light source input results in an output of light from the right light source 110R. Likewise, as an example, if the processor contains instructions for providing an auditory stimuli to the left ear of the user, then the processor can generate a left auditory source input. The generation of a left auditory source input can result in an output of sound from the left auditory source 120L. As another example, if the processor contains instructions for providing an auditory stimuli to the right ear of the user, then the processor can generate a right auditory source input. Thus, the generation of a right auditory source input can result in an output of sound from the right auditory source 120R.

The left light source 110L, using the left light source input, can display a left visual stimulus pattern. The right light source 110R, using a right light source input, can display a right visual stimulus pattern. The left auditory source 120L, using a left auditory source input, can display a left auditory stimulus pattern. The right auditory source 120R, using a right auditory source input, can display a right auditory stimulus pattern based upon the right auditory source input.

Additionally, if the processor receives instructions from the controller 130 for providing tactile stimuli to the left side of the user's head, then the processor can coordinate the output of a vibration from the left tactile source 121L. Likewise, if the processor executes instructions for providing tactile stimuli to the right side of the user's head, the headset 140 can coordinate the output of a vibration from the right tactile source 121R.

In a step 520, the headset 140 can be coupled to the head of the user. The coupling step 520 can be accomplished through positioning the frame of the headset 140 on the head of the user in a fixed position. In some instances, the frame can be positioned atop, on, or around the ears of the user. In other instances, the frame can be positioned, in part, over the top of the head of the user to secure the frame in a stable position around the head of the user.

In a step 530, the processor receives programming 450 with instructions to execute sensory stimulation by the headset 140 for treating the user. A controller 130 can be used to transmit the sensory stimulation instructions to the headset 140. The controller can be a control system operatively coupled to the headset, which can control functions of the headset 140 remotely. The controller 130 can be any control system operable by a doctor, nurse, or therapist providing the treatment to the user, or by any other desired person. For example, the controller 130 can be the control system within one or more of a personal computer, a laptop computer, a tablet computer, a smartphone, a wearable computer, or any other desired computing system.

The programming 450 can include instructions for providing stimuli to the user. For example, the programming 450 can include subroutine 450a, which includes instructions for the simultaneous activation of all auditory, video, and/or tactile stimulus sources. Another example programming 450 can include subroutine 450b, which includes instructions for alternating the left auditory, video, and/or tactile stimulus sources with the right auditory, video, and/or tactile stimulus sources, respectively (i.e., the left stimuli and right stimuli alternate being active and inactive). Another example programming 450 can include subroutine 450c, which includes alternating the visual sources with the auditory and/or tactile sources (i.e., the visual stimuli and the auditory/tactile stimuli alternate being active and inactive). Additionally, as an example, the programming 450 can include subroutine 450d, which includes alternating the left auditory and/or tactile source and the right visual source with the right auditory and/or tactile source and the left visual source (i.e., opposite auditory/tactile stimuli alternate being active and inactive). The programming 450 can include any desired combinations of the subroutines 450a, 450b, 450c, and 450d.

The processor of the headset 140 can be operatively coupled to the controller 130 to receive instructions for providing stimuli in the form of programming 450. For example, the processor can receive the programming 450 from the controller 130 and execute the programming 450 using the headset 140 130. In some embodiments, the controller 130 can include a pre-determined set of programming known to treat PTSD. In other embodiments, the controller 130 can be programmed in real-time or near real-time based on the needs of the user. The programming 450 includes instructions for the processor to execute two or more of auditory, video, and/or tactile stimulus, which can be activated, in real-time or near real-time, by headset 140 to the user.

In a step 540, responsive to receiving the sensory stimulation, the headset 140 administers a therapeutically effective amount of the sensory stimulation to the user. As described and disclosed herein, a therapeutically effective amount of sensory stimulation is an amount of stimuli that stimulates the brain of a user in a way that causes communication between the left and right sides of the brain. Accordingly, a “therapeutically effective amount” correlates with an amount of sensory stimulation that lead to one or more desired effects, such as, one or more therapeutic effects or other beneficial effects. A therapeutically effective amount of a sensory stimulation can vary according to factors such as the neurological disorder, age, sex, weight of the user, any other factor, or combination thereof. Providing the therapeutically effective amount of sensory stimulation to the brain of the user may cause output of the left and right channels of the brain and result in the left and right sides of the brain communicating with each other. This forced communication, for example, can allow PTSD memories to be wired to both sides of the brain. By wiring PTSD memories to both sides of the brains, sudden onset flashbacks of traumatic memories may be reduced or entirely eliminated. The reduction of traumatic memory occurrences may increase a user's ability to sleep through the night thereby improving the user's vital health statistics, for example, blood pressure, pulse rate, or any other vital health statistic. Further, providing different pulse frequency stimuli at a therapeutically effective amount may reduce flashbacks, nightmares, and hypervigilance in the user.

The headset 104 can administer the therapeutically effective amount of treatment through various auditory, visual, and/or tactile patterns. Specifically, a left visual stimulus pattern refers to a pattern of cycling between light and no light using a left light source 110L, a right visual stimulus pattern refers to a pattern of cycling between light and no light using a right light source 110R, a left auditory stimulus pattern refers to a pattern of cycling between silence and sound using a left auditory source 120L, a right auditory stimulus pattern refers to a pattern of cycling between silence and sound using a right auditory source 120R, a left tactile stimulus pattern refers to a pattern of vibrations using the right tactile source 121L, a right tactile stimulus pattern refers to a pattern of vibrations using the left tactile source 121R, any other desired pattern or combination thereof. Each of the various stimulus patterns can operate a different pulse rate and frequency from the other stimulus patterns. In some embodiments, the stimulus patterns operate at the same frequency and/or pulse rate.

The sensory stimulation administered at step 540 can alternate between the processes of a first set of sensory stimulation instructions (known as the first sensory stimulation) and a second set of sensory stimulation instructions (known as the second sensory stimulation). For example, the first sensory stimulation can include simultaneously providing a left visual stimulus pattern to a left eye of the user and a right auditory stimulus pattern to a right side of the head of the user. In this example, the second sensory stimulation can include simultaneously providing a right visual stimulus pattern to a right eye of the user and a left auditory stimulus pattern to a left side of the head of the user.

The first sensory stimulation and the second sensory stimulation can include a first stimulus pattern having a first pulse frequency, a second stimulus pattern having a second pulse frequency, and a third stimulus pattern having a third pulse frequency. In some instances, one of the first pulse frequency, the second pulse frequency, or the third pulse frequency is at or between 3.75 Hz and 4.25 Hz. In other instances, the first pulse frequency is at or between 7.5 Hz and 8.5 Hz, the second pulse frequency is at or between 2.5 Hz and 3.5 Hz, and the third pulse frequency is at or between 0.5 Hz and 1.5 Hz.

As discussed above and herein, the left auditory and tactile sources 120L and 121L and the right auditory and tactile sources 120R and 121R can each comprise bone conduction transducer that can provide both auditory and tactile stimulus.

In certain embodiments, the headset 140 provides two or more of auditory, video, and/or tactile stimulus concurrently, which can result in improved therapeutic benefits as compared to providing only one of auditory, visual, or tactile stimulus at one time. The two or more auditory, video, and/or tactile stimulus may thus combine to provide the improved therapeutic benefits, for example (i.e., the two or more auditory, video, and/or tactile stimulus may synergize in a way to provide improved results over providing two of the stimuli individually.)

The administration of the sensory stimulation at step 540 can occur for a pre-determined treatment period. As shown in Table 4 of Example 5, discussed below, the pre-determined treatment period (i.e., the amount of time that the device is set to be used) may be approximately 5 hours. In other instances, also shown in Table 4 of Example 5, the pre-determined treatment period may be approximately 2 hours. The pre-determined treatment period may be less than 2 hours, at or between 2 to 5 hours, or more than 5 hours. The time of the pre-determined treatment period may vary for each treatment session of the user or the pre-determined treatment period may be a similar time period for each treatment session. For example, as a user exhibits improved health, the pre-determined treatment period may lessen.

Finally, at step 550, after the headset 104 administers the therapeutically effective amount of the sensory stimulation to the user, the headset 104 collects a set of user data. The set of user data can include data collected from data sensors present or connected to the headset. The data sensors, for example, can collect user data including a pulse rate, a body temperature, a respiration rate, a blood pressure, any other desired data, or combinations thereof. Further, the collected set of user data can include reported data from the user, including information on sleep schedule, frequency of PTSD-related memories or flashbacks, severity of PTSD-related memories of flashback, anxiety, or combinations thereof.

Although the above steps of method 500 for treating a patient in accordance with one or more embodiments of the present disclosure, a person of ordinary skill in the art will recognize many variations based on the teaching described herein. The steps can be completed in a different order. Steps may be added or deleted. Some of the steps may comprise sub-steps. Many of the steps can be repeated as often as beneficial to the treatment of the user.

One or more of the steps of the method 500 may be performed with the circuitry as described herein, for example, circuitry of the controller 130 or the external control unit 130a such as logic circuitry such as a central processing unit (CPU) or a programmable array logic for field programmable gate array. The circuitry may be programmed to provide one or more of the steps of the method 500, and the program can comprise program instructions stored on a computer readable memory or programmed steps of the logic circuitry such as the programmable array logic or the field programmable gate array, for example.

### Example 1

The following describes an example of a stimulation pattern that has been found by empirical studies to be effective for inducing sleep, including napping, increasing neuroplasticity, treating brain injuries from strokes, TBI, or mTBI, improving balance, including improving fine motor control and reaction times, and treating PTSD, to name a few indications.

Light and auditory stimulus at a first frequency may be provided for a first time segment, then at a second lower frequency for a second time segment, and then at a third lower frequency for a third time segment. Each time segment may include one or more sub-segments of light and auditory stimulus, each sub-segment comprising one of the subroutines described above, for example. The light and auditory stimulus may end after a pre-determined time period, such as 20 minutes. The light and auditory stimulus may be ramped back up (i.e., starting from the third frequency, then transitioning to the second frequency, and finally transitioning to the third frequency), such as to wake the user. Alternatively or in combination, the light and auditory stimulus may be maintained at the second frequency such as to maintain a sleep state of the user. As described above, tactile stimulus may be provided concurrently with the auditory stimulus. The light may be provided at a wavelength of 580 nm and the auditory having a frequency of 256 Hz may be provided, or any of a number of auditory frequencies or combinations thereof that the subject can select as they wish.

Table 1 below describes an exemplary treatment regimen for this example. The stimulation provided in Table 1 first cycles through a block of four Segment A outputs, then cycles through a block of four Segment B outputs, then cycles through seven blocks of four Segment C outputs, and lastly repeats the block of four Segment A outputs. For Segment A outputs (A1, A2, A3, and A4), the auditory and light outputs cycle 115 or 116 times between being on for 0.1277 seconds and then being off for 0.1277 seconds (that is, at a pulse frequency of 3.9 Hz), followed by no output for 0.5 seconds. For Segment B outputs (B1, B2, B3 and B4), the auditory and light outputs cycle 44 or 45 times between being on for 0.3333 seconds and then being off for 0.3333 seconds (that is, at a pulse frequency of 1.5 Hz) followed by no output for 0.5 seconds. For Segment C outputs (C1, C2, C3 and C4), the auditory and light outputs cycle 14 or 15 times between being on for 1 second and then being off for 1 second (that is, a pulse frequency of 0.5 Hz), followed by no output for 1 second. Segments A1, B1, and C1 pulse the right and left sides of both the light and auditory together, with all outputs are synchronized to be on or off at the same time, as provided by subroutine 450a. Segments A2, B2, and C2 synchronize the left side light and auditory output, and the right side light and auditory output to be opposite to one another, as provided by subroutine 450b. Segments A3, B3, and C3 synchronize both lights together to be opposite to both auditory outputs, as provided by subroutine 450c. Segments A4, B4, and C4 synchronize the right auditory and light to be opposite to the left auditory and light outputs, as provided by subroutine 450d.

TABLE 1

| | Auditory | Auditory | | |
|---|---|---|---|---|
| | Left | Right | Light Left | Light Right |
| Segments A1-A4 for 120 s | | | | |
| Segment A1 (Light and Auditory | On 0.1277 s | On 0.1277 s | On 0.1277 s | On 0.1277 s |
| both sides pulse together) | | | | |
| Repeat 116 times, followed by | Off 0.1277 s | Off 0.1277 s | Off 0.1277 s | Off 0.1277 s |
| 0.5 sec gap | | | | |
| Segment A2 (light and auditory | On 0.1277 s | Off 0.1277 s | On 0.1277 s | Off 0.1277 s |
| on left side, alternating light and | | | | |
| auditory on Right). | | | | |
| Repeat 116 times, followed by | Off 0.1277 s | On 0.1277 s | Off 0.1277 s | On 0.1277 s |
| 0.5 sec gap | | | | |
| Segment A3 (both lights | On 0.1277 s | On 0.1277 s | Off 0.1277 s | Off 0.1277 s |
| together, alternating with both | | | | |
| auditories together) | | | | |
| Repeat 115 times, followed by | Off 0.1277 s | Off 0.1277 s | On 0.1277 s | On 0.1277 s |
| 0.5 sec gap | | | | |
| Segment A4 (auditory left and | On 0.1277 s | Off 0.1277 s | Off 0.1277 s | On 0.1277 s |
| light right together, alternating | | | | |
| auditory right and light left | | | | |
| together) | | | | |
| Repeat 115 times, followed by | Off 0.1277 s | On 0.1277 s | On 0.1277 s | Off 0.1277 s |
| 0.5 sec gap | | | | |
| Segments B1-B4 for 120 s | | | | |
| Segment B1 (Light and | On 0.3333 s | On 0.3333 s | On 0.3333 s | On 0.3333 s |
| Auditory both sides pulse | | | | |
| together) | | | | |
| Repeat 45 times, followed by 0.5 | Off 0.3333 s | Off 0.3333 s | Off 0.3333 s | Off 0.3333 s |
| sec gap | | | | |
| Segment B2 (light and auditory | On 0.3333 s | Off 0.3333 s | On 0.3333 s | Off 0.3333 s |
| on left side, alternating light and | | | | |
| auditory on Right) | | | | |
| Repeat 44 times, followed by | Off 0.3333 s | On 0.3333 s | Off 0.3333 s | On 0.3333 s |
| 0.5 sec gap | | | | |
| Segment B3 (both lights | On 0.3333 s | On 0.3333 s | Off 0.3333 s | Off 0.3333 s |
| together, alternating with both | | | | |
| auditories together) | | | | |
| Repeat 44 times, followed by 0.5 | Off 0.3333 s | Off 0.3333 s | On 0.3333 s | On 0.3333 s |
| sec gap | | | | |
| Segment B4 (auditory left and | On 0.3333 s | Off 0.3333 s | Off 0.3333 s | On 0.3333 s |
| light right together, alternating | | | | |
| auditory right and light left | | | | |
| together) | | | | |
| Repeat 44 times, followed by 0.5 | Off 0.3333 s | On 0.3333 s | On 0.3333 s | Off 0.3333 s |
| sec gap | | | | |
| Repeat the following Segments | | | | |
| C1-C4 7 times for a total of 14 | | | | |
| minutes | | | | |
| Segment C1 (Light and Auditory | On 1 sec | On 1 sec | On 1 sec | On 1 sec |
| both sides pulse together) | | | | |
| Repeat 15 times, followed by 1 | Off 1 sec | Off 1 sec | Off 1 sec | Off 1 sec |
| sec gap | | | | |
| Segment C2 (light and auditory | On 1 sec | Off 1 sec | On 1 sec | Off 1 sec |
| on left side, alternating light and | | | | |
| auditory on Right) | | | | |
| Repeat 15 times, followed by 1 | Off 1 sec | On 1 sec | Off 1 sec | On 1 sec |
| sec gap | | | | |
| Segment C3 (both lights | On 1 sec | On 1 sec | Off 1 sec | Off 1 sec |
| together, alternating with both | | | | |
| auditories together) | | | | |
| Repeat 14 times, followed by 1 | Off 1 sec | Off 1 sec | On 1 sec | On 1 sec |
| sec gap | | | | |
| Segment C4 (auditory left and | On 1 sec | Off 1 sec | Off 1 sec | On 1 sec |
| light right together, alternating | Off 1 sec | On 1 sec | On 1 sec | Off 1 sec |
| auditory right and light left | | | | |
| together) | | | | |
| Repeat 14 times, followed by 1 | Off 1 sec | On 1 sec | On 1 sec | Off 1 sec |
| sec gap | | | | |
| Segments A1-A4 for 120 s | | | | |
| Segment A1 (Light and Auditory | On 0.1277 | On 0.1277 | On 0.1277 | On 0.1277 |
| both sides pulse together) | | | | |
| Repeat 116 times, followed by | Off 0.1277 | Off 0.1277 | Off 0.1277 | Off 0.1277 |
| 0.5 sec gap | | | | |
| Segment A2 (light and auditory | On 0.1277 | Off 0.1277 | On 0.1277 | Off 0.1277 |
| on left side, alternating light and | | | | |
| auditory on Right) | | | | |
| Repeat 116 times, followed by | Off 0.1277 | On 0.1277 | Off 0.1277 | On 0.1277 |
| 0.5 sec gap | | | | |
| Segment A3 ( both lights | On 0.1277 | On 0.1277 | Off 0.1277 | Off 0.1277 |
| together, alternating with both | | | | |
| auditories together) | | | | |
| Repeat 115 times, followed by | Off 0.1277 | Off 0.1277 | On 0.1277 | On 0.1277 |
| 0.5 sec gap | | | | |
| Segment A4 (auditory left and | On 0.1277 | Off 0.1277 | Off 0.1277 | On 0.1277 |
| light right together, alternating | | | | |
| auditory right and light left | | | | |
| together) | | | | |
| Repeat 115 times, followed by | Off 0.1277 | On 0.1277 | On 0.1277 | Off 0.1277 |
| 0.5 sec gap | | | | |

### Example 2

The following describes an example of a stimulation pattern that has been found by empirical studies to be effective for inducing sleep. The stimulation pattern of Example 2 includes the part of the treatment regimen shown in Table 1. Specifically, the stimulation first cycles through a block of four Segment A outputs, then cycles through a block of four Segment B outputs, and then cycles through seven blocks of four Segment C outputs. The repetition of the last block of four Segment A outputs is not provided in Example 2.

### Example 3

The following described example of a stimulation pattern that has been found by empirical studies to be effective for increasing alpha wave brain activity, inducing neuroplasticity, treating stroke or other brain injuries such as TBI, mTBI, including improving balance, improving fine motor control and reaction times, and treating PTSD, to name a few indications.

In this example, the four subroutines described above and herein are applied and repeated for multiple time segments, each at a predetermined stimulation (repetition) frequency. The four subroutines may be repeated, such as with each segment of the four subroutines lasting 120 seconds, for example. As described above, tactile stimulus may be provided concurrently with the auditory stimulus. The light may be provided at a wavelength of 580 nm and the auditory having a frequency of 432 Hz may be provided.

Table 2 below describes an exemplary treatment regimen for this example. The stimulation provided in Table 2 cycles through a block of four Segment A outputs 10 times. For Segment A1, A2, A3, and A4, the auditory and light outputs cycle 115 or 116 times between being on for 0.1277 seconds and then being off for 0.1277 seconds, followed by no output for 0.5 seconds. Segments A1 pulses the right and left sides of both the light and auditory together, with all outputs are synchronized to be on or off at the same time, as provided by subroutine 450a. Segment A2 synchronizes the left side light and auditory output, and the right side light and auditory output to be opposite to one another, as provided by subroutine 450b. Segment A3 synchronizes both lights together to be opposite to both auditory outputs, as provided by subroutine 450c. Segment A4 synchronizes the right auditory and light to be opposite to the left auditory and light outputs, as provided by subroutine 450d.

TABLE 2

| | | Auditory | | |
|---|---|---|---|---|
| | Auditory Left | Right | Light Left | Light Right |
| Repeat the following Segments | | | | |
| A1-A4 10 times for a total time | | | | |
| of 20 minutes | | | | |
| Segment A1 (Light and Auditory | On 0.1277 s | On 0.1277 s | On 0.1277 s | On 0.1277 s |
| both sides pulse together) | | | | |
| Repeat 116 times, followed by | Off 0.1277 s | Off 0.1277 s | Off 0.1277 s | Off 0.1277 s |
| 0.5 sec gap | | | | |
| Segment A2 (light and auditory | On 0.1277 s | Off 0.1277 s | On 0.1277 s | Off 0.1277 s |
| on left side, alternating light and | | | | |
| auditory on Right) | | | | |
| Repeat 116 times, followed by | Off 0.1277 s | On 0.1277 s | Off 0.1277 s | On 0.1277 s |
| 0.5 sec gap | | | | |
| Segment A3 ( both lights | On 0.1277 s | On 0.1277 s | Off 0.1277 s | Off 0.1277 s |
| together, alternating with both | | | | |
| auditories together) | | | | |
| Repeat 115 times, followed by | Off 0.1277 s | Off 0.1277 s | On 0.1277 s | On 0.1277 s |
| 0.5 sec gap | | | | |
| Segment A4 (auditory left and | On 0.1277 s | Off 0.1277 s | Off 0.1277 s | On 0.1277 s |
| light right together, alternating | | | | |
| auditory right and light left | | | | |
| together) | | | | |
| Repeat 115 times, followed by | Off 0.1277 s | On 0.1277 s | On 0.1277 s | Off 0.1277 s |
| 0.5 sec gap | | | | |

### Example 4

The following described yet another example of a stimulation pattern that has been found by empirical studies to be effective for increasing energy levels in the subject. Light and auditory stimulus at a first frequency may be provided for a first time segment, then at a second higher frequency for a second time segment, then back at the first frequency for a subsequent time segment, and so forth. Each time segment may include one or more sub-segments of light and auditory stimulus, each sub-segment comprising one of the subroutines described above, for example. The light and auditory stimulus may end after a pre-determined time period, such as 20 minutes. As described above, tactile stimulus may be provided concurrently with the auditory stimulus. The light may be provided at a wavelength of 580 nm and the auditory having a frequency of 432 Hz may be provided.

Table 3 below describes an exemplary treatment regimen for this example. The stimulation provided in Table 3 cycles ten times first through a block of four Segment A outputs, then through a block of four Segment D outputs. For Segment A outputs (A1, A2, A3, and A4), the auditory and light outputs cycle 115 or 116 times between being on for 0.1277 seconds and then being off for 0.1277 seconds, followed by no output for 0.5 seconds. For Segment D outputs (D1, D2, D3 and D4), the auditory and light outputs cycle 44 or 45 times between being on for 0.0667 seconds and then being off for 0.0667 seconds, followed by no output for 0.5 seconds. Segments A1 and D pulse the right and left sides of both the light and auditory together, with all outputs are synchronized to be on or off at the same time, as provided by subroutine 450a. Segments A2 and D2 synchronize the left side light and auditory output, and the right side light and auditory output to be opposite to one another, as provided by subroutine 450b. Segments A3 and D3 synchronize both lights together to be opposite to both auditory outputs, as provided by subroutine 450c. Segments A4 and D4 synchronize the right auditory and light to be opposite to the left auditory and light outputs, as provided by subroutine 450d.

TABLE 3

| | Auditory | | | |
|---|---|---|---|---|
| | Left | Auditory Right | Light Left | Light Right |
| Repeat 10 times: Segments | | | | |
| A1-A4 followed by Segments | | | | |
| D1-D4, for a total time of 20 | | | | |
| minutes | | | | |
| Segment A1 (Light and | On 0.1277 s | On 0.1277 s | On 0.1277 s | On 0.1277 s |
| Auditory both sides pulse | | | | |
| together) | | | | |
| Repeat 116 times, followed by | Off 0.1277 s | Off 0.1277 s | Off 0.1277 s | Off 0.1277 s |
| 0.5 sec gap | | | | |
| Segment A2 (light and | On 0.1277 s | Off 0.1277 s | On 0.1277 s | Off 0.1277 s |
| auditory on left side, | | | | |
| alternating light and auditory | | | | |
| on Right) | | | | |
| Repeat 116 times, followed by | Off 0.1277 s | On 0.1277 s | Off 0.1277 s | On 0.1277 s |
| 0.5 sec gap | | | | |
| Segment A3 (both lights | On 0.1277 s | On 0.1277 s | Off 0.1277 s | Off 0.1277 s |
| together, alternating with both | | | | |
| auditories together) | | | | |
| Repeat 115 times, followed by | Off 0.1277 s | Off 0.1277 s | On 0.1277 s | On 0.1277 s |
| 0.5 sec gap | | | | |
| Segment A4 (auditory left and | On 0.1277 s | Off 0.1277 s | Off 0.1277 s | On 0.1277 s |
| light right together, alternating | | | | |
| auditory right and light left | | | | |
| together) | | | | |
| Repeat 115 times, followed by | Off 0.1277 s | On 0.1277 s | On 0.1277 s | Off 0.1277 s |
| 0.5 sec gap | | | | |
| Segment D1 (Light and | On 0.0667 s | On 0.0667 s | On 0.0667 s | On 0.0667 s |
| together) | | | | |
| Auditory both sides pulse | | | | |
| Repeat 221 times, followed by | Off 0.0667 s | Off 0.0667 s | Off 0.0667 s | Off 0.0667 s |
| 0.5 sec gap | | | | |
| Segment D2 (light and | On 0.0667 s | Off 0.0667 s | On 0.0667 s | Off 0.0667 s |
| auditory on left side, | | | | |
| alternating light and auditory | | | | |
| on Right) | | | | |
| Repeat 221 times, followed by | Off 0.0667 s | On 0.0667 s | Off 0.0667 s | On 0.0667 s |
| 0.5 sec gap | | | | |
| Segment D3 (both lights | On 0.0667 s | On 0.0667 s | Off 0.0667 s | Off 0.0667 s |
| together, alternating with both | | | | |
| auditories together) | | | | |
| Repeat 221 times, followed by | Off 0.0667 s | Off 0.0667 s | On 0.0667 s | On 0.0667 s |
| 0.5 sec gap | | | | |
| on Right) | | | | |
| Segment D4 (auditory left and | On 0.0667 s | Off 0.0667 s | Off 0.0667 s | On 0.0667 s |
| light right together, alternating | | | | |
| auditory right and light left | | | | |
| together) | | | | |
| Repeat 221 times, followed by | Off 0.0667 s | On 0.0667 s | On 0.0667 s | Off 0.0667 s |
| 0.5 sec gap | | | | |

### Example 5

The following Table 4 lists experimental results for the use of the inventive methods. The table lists what was being tested or treated, details of the conditions, the number of subjects, and the results of the tests. In each case, the stimulation in Example 1 for treating non-sleep related problems and for inducing a short sleep, and the stimulation in Example 2 was used for all other treatments.

Several of the treatments provided improvements in physical and/or mental performance, such as improving fine motor control and reaction times. This may be due to the device providing improved neuroplasticity in the days after treatment. Other treatments provided improvements in performing tasks and recovery from brain injury, such as injuries resulting from oxygen deprivation (strokes) and for those suffering from traumatic brain injury (TBI) or mild traumatic brain injury, and my provide improving balance, improving fine motor control. Other treatments provided relief to sufferers of PTSD by reducing the subject's response to triggering stimuli.

TABLE 4

| | | No. of | |
|---|---|---|---|
| Treatment For | Details | subjects | Results |
| Pain | Reduction of chronic | 1 | Eliminated chronic nerve damage |
| Management | nerve damage pain and | | pain for the time the device was |
| | improvement of sleep on | | used. |
| | self. Use of device for 3 | | |
| | months with 20 min/day of | | |
| | use of device. | | |
| PTSD | Treating PTSD. Device use time | 3 | Reduced flashbacks, nightmares and |
| | of 5 hours. | | hypervigilance in all 3 subjects |
| Performance | Marksmanship (rifles and | \|20 | Significant improvements in |
| Enhancement | pistols), endurance and speed | | Imarksmanship in all participants and |
| | driving (advanced surveillance, | | ease of concentration during speed |
| | coordination and evasion). 6 | | driving, faster times on endurance |
| | hours training each subject. | | trials for 19/20 subjects |
| Performance | Fine motor skills on bomb | 3 | Improved performance of fine motor |
| Enhancement | disposal personnel 3 hours | | skills on bomb disposal VR simulation |
| | training with device. | | for all subjects |
| Performance | Fine motor skills of surgeons- | 3 | Improved performance of fine motor |
| Enhancement | 3 hours training each. | | skills on surgical procedures VR |
| | | | simulation for all subjects. |
| Performance | Pistol use and marksmanship. 3 | 2 | 10% and 30% respectively |
| Enhancement | hours training. | | increased speed in stripping and |
| | | | reassembling weapons. (average |
| | | | each of 5 tests, pre and post |
| | | | training) 6% average |
| | | | improvement in marksmanship |
| | | | scores-highly significant for |
| | | | such level of skill for all subjects |
| Performance | Performance by anti-terror and | 5 | 10% average improvement in scores. |
| Enhancement | anti-drug squads of an elite | | Total absence of any PTSD |
| and PTSD | firearms unit of a police force. 3 | | |
| | hours training each. | | |
| Performance | Marksmanship. 2 hours training. | 1 | Average grouping shrunk from 5 inches |
| Enhancement | | | to 1 inch at 200 yds. |
| Brain State | Increasing alpha activity. 4 | 20 | Results as predicted. Group 1 greatest |
| | hours total training time per | | change, followed by group 2, Group 3 |
| | subject. Group 1: L & S | | least change of active groups. Group 4 |
| | stimulation and biofeedback. | | no change. |
| | Group 2: just L & S stimulation | | |
| | Group 3: just biofeedback, | | |
| | Group 4: control. Double | | |
| | blinded; those administering | | |
| | had no idea of what was | | |
| | predicted to happen. | | |
| Performance | Marksmanship. | 3 + 15 | Significant improvement for all |
| Enhancement | | | subjects. |
| Mental | Attention, learning and | 3 | positive reports from all subjects |
| Performance | resistance to interrogation. 4 | | |
| Enhancement | hours each person. Conduct | | |
| | after Capture course. | | |
| Performance | Motion sickness for fixed wing | 4 | Dramatic improvements in half of |
| Enhancement | aircraft pilots who have | | subjects. Small improvements in |
| | developed problems. 4 hours | | remaining half of subjects |
| | training per subject. | | |
| PTSD | PTSD symptoms: Test to | 33 | Successful in 31/33 subjects |
| | remove neurological | | |
| | symptoms of flashbacks, | | |
| | nightmares and cold sweats. | | |
| Performance | Driver performance using VR | 2 | Immediate increase in reaction |
| Enhancement | simulators for reaction speeds | | speeds and improved |
| | and performance under stress. | | performance for all subjects |
| Performance | Professional soccer player | 1 | 5-25% increase in speeds to complete |
| Enhancement | performance. Trained for 4 | | tests |
| | hours. Battery of 21 tests. | | |
| Inducing Sleep | Sleep patterning and circadian | 6 | All subjects fell asleep using the device |
| | rhythm adjustment for crews | | during training, including one subject |
| | setting endurance records. | | that was ill with a virus and couldn't |
| | Members each year. Also used | | otherwise sleep. |
| | for improving safety drills when | | |
| | parachuting. | | |
| Performance | Race car driver performance. | 1 | Subject won his first Grand Prix of the |
| Enhancement | Ten days of training for 30 | | season. |
| | minutes per day. | | |
| Performance | Soccer player kicking | 1 | Subject went from 5th ranked to |
| Enhancement | performance. 5 days of 1 hour each day. | | highest ranked |
| Stroke | Use on 6 year post stroke | 10 | Observable balance improvement in |
| Recovery | subjects. 4 hours training. | | 7/10 subjects. 3 subjects had had |
| | | | dramatic improvements in their |
| | | | sleep. |
| Epilepsy | Effect on seizures of | 3 | One subject was found to not be |
| Seizure | photosensitive epileptics. 4 | | epileptic. The other two subjects had a |
| Reduction | hours training. | | reduction in both severity and |
| | | | frequency of seizures, for at least a |
| | | | period of at least one month. |
| Concussion | Effect on concussions. | 18 | All subjects appeared to have recovery |
| Recovery | | | happen at very fast speed. |
| Performance | Effect on musical ability of a | 1 | Greatly improved performance speed |
| Enhancement | jazz musician. | | |
| PTSD | PTSD. Treatment protocol | 22 | 19 individuals saw a cessation of |
| | lasting 3 sessions of 2 hours | | major symptoms - flashbacks, |
| | each. | | nightmares, cold sweats and |
| | | | hypervigilance. the remaining 3 |
| | | | appeared to be calmer after treatment, |
| | | | but did not stop the major neurological |
| | | | symptoms |
| Sleep | Insomnia. | 1 | Goes to sleep 4 times in 45 mins |
| Pain | Chronic Regional Pain | 1 | Subject had constant pain on touching |
| Management | Syndrome. | | arms with no relief in 3 years Subject |
| | | | saw immediate pain relief on first use |
| | | | of the device. Continued use over the |
| | | | following weeks resulted in periods of |
| | | | time without pain increase up to four |
| | | | hours following each use. Averaging at |
| | | | two hours. |
| Pain | Chronic pain. | 1 | After six months of use, the subject |
| Management | | | continues getting 30% more sleep, and |
| and Sleep | | | a significant reduction in pain. Device |
| | | | continues to be used 3-4 times a week |
| | | | for 20 min. |

### Example Embodiments

The following are example embodiments.

An example embodiment 1 comprises a method of providing stimulation to a user, the method comprises: providing a headset to be worn by the user; applying, with the headset, a left visual stimulus pattern to the left eye of the user; applying, with the headset, a right visual stimulus pattern to the right eye of the user; applying, with the headset, a left auditory stimulus pattern to the left side of a head of the user; and applying, with the headset, a right auditory stimulus pattern to the right side of the head, wherein the applications of the left visual stimulus pattern, the right visual stimulus pattern, the left auditory stimulus pattern, and the right auditory stimulus pattern are coordinated with one another.

An example embodiment 2 including example embodiment 1, wherein applying, with the headset, the left auditory stimulus pattern comprises applying, with the headset, a left tactile stimulus pattern, and wherein applying, with the headset, the right auditory stimulus pattern comprises applying, with the headset, a right tactile stimulus pattern.

An example embodiment 3 including example embodiment 2, wherein the left tactile stimulus pattern and the right tactile stimulus pattern are configured to produce a plurality of concurrent left and right tactile signals.

An example embodiment 4 including example embodiment 2, wherein the left tactile stimulus pattern and the right tactile stimulus pattern are configured to produce a plurality of alternating left and right tactile signals.

An example embodiment 5 including example embodiment 2, wherein the left tactile stimulus pattern is coordinated with the left auditory stimulus pattern, and wherein the right tactile stimulus pattern is coordinated with the right auditory stimulus pattern.

An example embodiment 6 including example embodiment 5, wherein the left tactile stimulus pattern comprises a left-side vibration at a first frequency generated concurrently with auditory during the left auditory stimulus pattern, and wherein the right tactile stimulus pattern comprises a right-side vibration at a second frequency generated concurrently with auditory during the right auditory stimulus pattern.

An example embodiment 7 including example embodiment 6, wherein one or more of the left-side or right-side vibration is a vibration of from 0.5 Hz to 1.5 Hz.

An example embodiment 8 including any one of example embodiments 1 through 7, wherein applying, with the headset, the left auditory stimulus pattern comprises generating the left tactile stimulus pattern with a left bone conduction transducer of the headset, and wherein applying, with the headset, the right auditory stimulus pattern comprises generating the left tactile stimulus pattern with a left bone conduction transducer of the headset.

An example embodiment 9 including any one of example embodiments 1 through 7, wherein the left visual stimulus pattern and the right visual stimulus pattern are configured to produce a plurality of concurrent left and right light signals.

An example embodiment 10 including any one of example embodiments 1 through 7, wherein the left visual stimulus pattern and the right visual stimulus pattern are configured to produce a plurality of alternating left and right light signals.

An example embodiment 11 including any one of example embodiments 1 through 7, wherein the left auditory stimulus pattern and the right auditory stimulus pattern are configured to produce a plurality of concurrent left and right auditory signals.

An example embodiment 12 including any one of example embodiments 1 through 7, wherein the left auditory stimulus pattern and the right auditory stimulus pattern are configured to produce a plurality of alternating left and right auditory signals.

An example embodiment 13 including any one of example embodiments 1 through 12, wherein one or more of the left or right visual stimulus pattern has a light wavelength of from 550 nm to 610 nm.

An example embodiment 14 including any one of example embodiments 1 through 13, wherein one or more of the left or right visual stimulus pattern has a light wavelength of 580 nm.

An example embodiment 15 including any one of example embodiments 1 through 14, wherein one or more of the left or right auditory stimulus pattern includes an auditory frequency of from 240 Hz to 480 Hz.

An example embodiment 16 including any one of example embodiments 1 through 15, wherein one or more of the left or right auditory stimulus pattern includes an auditory frequency of 256 Hz or 432 Hz.

An example embodiment 17 including any one of example embodiments 1 through 16, wherein one or more of the left visual stimulus pattern comprises repeatedly pulsing a light at one or more of a first frequency, a second frequency less than the first frequency, or a third frequency less than the first and second frequencies.

An example embodiment 18 including any one of example embodiments 1 through 17, wherein the first frequency is between 3.75 Hz and 4.25 Hz, the second frequency is between 1.25 Hz and 1.75 Hz, and the third frequency is between 0.25 Hz and 0.75 Hz.

An example embodiment 19 including example embodiment 19, wherein the first frequency is 3.9 Hz, the second frequency is 1.5 Hz, and the third frequency is 1 Hz.

An example embodiment 20 including any one of example embodiments 18 and 19, wherein repeatedly pulsing the light comprises pulsing the light for a predetermined time interval.

An example embodiment 21 including example embodiment 20, wherein the predetermined time interval is 25-35 seconds.

An example embodiment 22 including any one of example embodiments 20 and 21, wherein the predetermined time interval is 30 seconds.

An example embodiment 23 including any one of example embodiments 1 through 22, wherein one or more of the left or right auditory stimulus pattern comprises a sequence stimulus patterns each having a pulse frequency having a pulse period, said repeating temporal signals including a portion of the pulse period with including an auditory frequency of from 240 Hz to 480 Hz and a portion of the pulse period.

An example embodiment 24 including example embodiment 23, wherein said portion of said pulse period is one half of the pulse period.

An example embodiment 25 including any one of example embodiments 23 and 24, wherein said sequence of stimulus patterns includes a first stimulus pattern having a first pulse frequency, a second stimulus pattern having a second pulse frequency less than the first pulse frequency, and a third stimulus pattern having a third pulse frequency less than the second pulse frequency.

An example embodiment 26 including example embodiment 25, wherein the first pulse frequency is between 3.75 Hz and 4.25 Hz, the second pulse frequency is between 1.25 Hz and 1.75 Hz, and the third pulse frequency is between 0.25 Hz and 0.75 Hz.

An example embodiment 27 including example embodiment 25 wherein the first pulse frequency is 3.9 Hz, the second pulse frequency is 1.5 Hz, and the third pulse frequency is 1 Hz.

An example embodiment 28 including any one of example embodiments 25 through 27, wherein said first stimulus pattern, said second stimulus pattern, or said third stimulus pattern stimulates for a predetermined time interval.

An example embodiment 29 including example embodiments 28, wherein the predetermined time interval is 25-35 seconds.

An example embodiment 30 including any one of example embodiments 28 and 29, wherein the predetermined time interval is 30 seconds.

An example embodiment 31 including any one of example embodiments 1 through 30, wherein the headset is in operative communication with an external control device.

An example embodiment 32 comprises a method of treating a neurological disease or condition or providing performance enhancement using the method of example embodiment 1.

An example embodiment 33 including example embodiment 32, where said neurological disease or condition comprises insomnia, PTSD, stokes or other brain injuries such as traumatic brain injury (TBI), or mild traumatic brain injury (mTBI).

An example embodiment 34 including example embodiment 32, where said performance enhancement is providing sleep, the improvement of mental capabilities, or the improvement of physical capabilities.

An example embodiment 35 including an apparatus to provide stimulation to a user, the apparatus comprises: a frame configured to be worn on a head of the user; a left light source configured to generate a left visual stimulus pattern; a right light source configured to generate a right visual stimulus pattern; a left auditory source configured to generate a left auditory stimulus pattern; a right auditory source configured to generate a right auditory stimulus pattern; and a controller coupled to the left light source, the right light source, the left auditory source, and the right auditory source, wherein applications of the left visual stimulus pattern, the right visual stimulus pattern, the left auditory stimulus pattern, and the right auditory stimulus pattern are independently controlled from one another but coordinated with one another by the controller.

An example embodiment 36 including example embodiment 35, wherein the left auditory source is further configured to generate a left tactile stimulus pattern, and wherein the right auditory source is further configured to generate a right tactile stimulus pattern.

An example embodiment 37 including any one of example embodiments 35 and 36, wherein one or more of the left or right auditory source comprises a bone conduction transducer.

An example embodiment 38 including any one of example embodiments 35 through 37, wherein the controller is configured to be in communication with and operated by an external control unit.

An example embodiment 39 including example embodiment 38, wherein the external control unit is in wireless communication with the controller.

An example embodiment 40 including any one of example embodiments 38 and 39, wherein the external control unit comprises one or more of a personal computer, a laptop computer, a tablet computer, a smartphone, or a wearable computer.

An example embodiment 41 including any one of example embodiments 38 through 40, wherein the external control unit has operating thereon an application configured to interface with and operate the controller.

An example embodiment 42 including any one of example embodiments 35 through 41, wherein one or more of the left or right light source comprises a light-emitting diode (LED).

An example embodiment 43 including any one of example embodiments 35 through 42, wherein one or more of the left or right light source is configured to generate light at 550-610 nm.

An example embodiment 44 including any one of example embodiments 35 through 42, wherein one or more of the left or right light source is configured to generate light at 580 nm.

An example embodiment 45 comprises a method to provide stimulation to a user, the method comprises: concurrently providing a left-side light stimulus to a left eye of the user, a right-side light stimulus to a right eye of the user, a left-side auditory stimulus to a left side of the user, and a right-side auditory stimulus to a right side of the user for a first time interval; alternating providing the left-side light stimulus and left-side auditory stimulus with providing the right-side light stimulus and right-side auditory stimulus for a second time interval; alternating providing the left-side and right-side light stimuli with providing the left-side and right-side auditory stimuli for a third time interval; and alternating providing the left-side light stimulus and right-side auditory stimulus with providing the right-side light stimulus and left-side auditory stimulus for a fourth time interval.

An example embodiment 46 including example embodiment 45, wherein the second time interval is after the first time interval, the third time interval is after the second time interval, and the fourth time interval is after the third time interval.

An example embodiment 47 including of any one of example embodiments 45 and 46, wherein one or more of the left-side or right-side light stimuli comprises pulsing a light at a predetermined pulsing frequency for one or more of the first, second, third, or fourth time intervals.

An example embodiment 48 including any one of example embodiments 45 through 47, wherein one or more of the left-side or right-side auditory stimuli comprises generating a auditory at a predetermined generation frequency for one or more of the first, second, third, or fourth time intervals.

An example embodiment 49 including any one of example embodiments 45 through 48, wherein the left-side light stimulus, the right-side light stimulus, the left-side auditory stimulus, and the right-side auditory stimulus are generated with a wearable headset.

An example embodiment 50 including any one of example embodiments 45 through 49, further comprises providing a left-side tactile stimulus concurrently with the left-side auditory stimulus and providing a right-side tactile stimulus concurrently with the right-side auditory stimulus.

An example embodiment 51 comprises a method of treating a neurological disease or condition or providing performance enhancement using the method of example embodiment 45.

An example embodiment 52 including example embodiment 51, where said neurological disease or condition comprises insomnia, PTSD, stokes or other brain injuries such as traumatic brain injury (TBI), or mild traumatic brain injury (mTBI).

An example embodiment 53 including example embodiment 51, where said performance enhancement is providing sleep, improving alpha wave activity, the improvement of mental capabilities, or the improvement of physical capabilities.

An example embodiment 54 comprises a method to provide stimulation to a user, the method comprises: providing a headset to be worn by the user; applying, with the headset, a left auditory stimulus pattern to the left side of a head of the user; and applying, with the headset, a right auditory stimulus pattern to the right side of the head, wherein the applications of the left auditory stimulus pattern and the right auditory stimulus pattern are coordinated with one another.

An example embodiment 55 including example embodiment 54, wherein the left auditory stimulus pattern and the right auditory stimulus pattern are configured to produce a plurality of concurrent left and right auditory signals.

An example embodiment 56 including example embodiment 54, wherein the left auditory stimulus pattern and the right auditory stimulus pattern are configured to produce a plurality of alternating left and right auditory signals.

An example embodiment 57 including any one of example embodiments 54 through 56, wherein one or more of the left or right auditory stimulus pattern includes an auditory frequency of from 240 Hz to 480 Hz.

An example embodiment 58 including any one of example embodiments 54 through 57, wherein one or more of the left or right auditory stimulus pattern includes an auditory frequency of 256 Hz or 432 Hz.

An example embodiment 59 including any one of example embodiments 54 through 58, wherein one or more of the left or right auditory stimulus pattern comprises a sequence stimulus patterns each having a pulse frequency having a pulse period, said repeating temporal signals including a portion of the pulse period with including an auditory frequency of from 240 Hz to 480 Hz and a portion of the pulse period.

An example embodiment 60 including example embodiment 59, wherein said portion of said pulse period is one half of the pulse period.

An example embodiment 61 including any one of example embodiments 59 and 60, wherein said sequence of stimulus patterns includes a first stimulus pattern having a first pulse frequency, a second stimulus pattern having a second pulse frequency less than the first pulse frequency, and a third stimulus pattern having a third pulse frequency less than the second pulse frequency.

An example embodiment 62 including example embodiment 61, wherein the first pulse frequency is between 3.75 Hz and 4.25 Hz, the second pulse frequency is between 1.25 Hz and 1.75 Hz, and the third pulse frequency is between 0.25 Hz and 0.75 Hz.

An example embodiment 63 including example embodiment 62, wherein the first pulse frequency is 3.9 Hz, the second pulse frequency is 1.5 Hz, and the third pulse frequency is 1 Hz.

An example embodiment 64 including any one of example embodiments 61 through 63, wherein said first stimulus pattern, said second stimulus pattern, or said third stimulus pattern stimulates for a predetermined time interval.

An example embodiment 65 including example embodiment 64, wherein the predetermined time interval is 25-35 seconds.

An example embodiment 66 including example embodiment 64, wherein the predetermined time interval is 30 seconds.

An example embodiment 67 including any one of example embodiments 54 through 66, wherein the headset is in operative communication with an external control device.

An example embodiment 68 comprises a method of treating a neurological disease or condition or providing performance enhancement using the method of example embodiment 54.

An example embodiment 69 including example embodiment 68, where said neurological disease or condition comprises insomnia, PTSD, or brain injuries such as traumatic brain injury (TBI), mild traumatic brain injury (mTBI), or strokes.

An example embodiment 70 including example embodiment 68, where said performance enhancement is providing sleep, the improvement of mental capabilities, or the improvement of physical capabilities.

An example embodiment 71 including method of treating a user having post-traumatic stress disorder (PTSD), including receiving a set of sensory stimulation instructions by a processor in a headset configured for being worn on a head of the user; responsive to receiving the set of sensory stimulation instructions, administering a therapeutically effective amount of a sensory stimulation to the user, where the sensory stimulation alternates between: a first sensory stimulation including simultaneously providing a left visual stimulus pattern to a left eye of the user and a right auditory stimulus pattern to a right side of the head, and a second sensory stimulation including simultaneously providing a right visual stimulus pattern to a right eye of the user and a left auditory stimulus pattern to a left side of the head of the user; wherein the first sensory stimulation and the second sensory stimulation include a first stimulus pattern having a first pulse frequency, a second stimulus pattern having a second pulse frequency, and a third stimulus pattern having a third pulse frequency; and collecting a set of user data associated with the administration of the therapeutically effective amount of the sensory stimulation to the user.

An example embodiment 72 including example embodiment 71, where one of the first pulse frequency, the second pulse frequency, or the third pulse frequency is at or between 3.75 Hz and 4.25 Hz, or the first pulse frequency is at or between 7.5 Hz and 8.5 Hz, the second pulse frequency is at or between 2.5 Hz and 3.5 Hz, and the third pulse frequency is at or between 0.5 Hz and 1.5 Hz.

An example embodiment 73 including example embodiment 71, where administering the therapeutically effective amount of the sensory stimulation to the user occurs for a pre-determined treatment period.

An example embodiment 74 including example embodiment 73, where the pre-determined treatment period is approximately 5 hours.

An example embodiment 75 including example embodiment 73, where the pre-determined treatment period is approximately 2 hours.

An example embodiment 76 including example embodiment 71, where the first sensory stimulation further includes providing a right tactile stimulus pattern to the right side of the head simultaneously with the left visual stimulus pattern and a right auditory stimulus pattern, and where the second sensory stimulation further includes providing a left tactile stimulus pattern to the left side of the head simultaneously with the right visual stimulus pattern and the left auditory stimulus pattern.

An example embodiment 77 including example embodiment 71, where the set of user data comprises at least one vital health statistic selected from the group consisting of a pulse rate, a body temperature, a respiration rate, and a blood pressure.

An example embodiment 78 including headset for providing a therapeutically effective amount of a sensory stimulation to a user, including: a frame configured to be worn on a head of the user; a processor configured to generate a plurality of inputs including a left light source input, a right light source input, a left auditory source input, and a right auditory source input; a left light source configured to produce, using the left light source input, a left visual stimulus pattern; a right light source configured to produce, using the right light source input, a right visual stimulus pattern; a left auditory source configured to produce, using the left auditory source input, a left auditory stimulus pattern; and a right auditory source configured to produce, using the right auditory source input, a right auditory stimulus pattern, where the processor is further configured to administer, by the headset, the therapeutically effective amount of the sensory stimulation to the user, where the plurality of inputs alternate between a first input including simultaneously generating the left light source input and the right auditory source input, and a second input including simultaneously generating the right light source input and the left auditory source input, and where the first input and the second input include a first stimulus pattern having a first pulse frequency, a second stimulus pattern having a second pulse frequency less than the first pulse frequency, and a third stimulus pattern having a third pulse frequency less than the second pulse frequency.

An example embodiment 79 including example embodiment 78, where one of the first pulse frequency, the second pulse frequency, or the third pulse frequency is at or between 3.75 Hz and 4.25 Hz, or the first pulse frequency is at or between 7.5 Hz and 8.5 Hz, the second pulse frequency is at or between 2.5 Hz and 3.5 Hz, and the third pulse frequency is at or between 0.5 Hz and 1.5 Hz.

An example embodiment 80 including example embodiment 78, where the processor is further programmed to provide the sensory stimulation for a pre-determined treatment period.

An example embodiment 81 including example embodiment 80, where the pre-determined treatment period is approximately 5 hours.

An example embodiment 82 including example embodiment 80, where the pre-determined treatment period is approximately 2 hours.

An example embodiment 83 including example embodiment 78, where the plurality of inputs further includes comprises a left tactile source input and a right tactile source input, and further includes a left tactile source configured to produce, using the left tactile source input, a left tactile stimulus pattern; and a right tactile source configured to produce, using the right tactile source input, a right tactile stimulus pattern, where the first input further includes generating the right tactile source input simultaneously with the left visual source input and the right auditory source input, and the second input further includes generating the left tactile source input simultaneously with the right visual source input and the left auditory source input.

An example embodiment 84 including example embodiment 78, further including data sensors, wherein the data sensors are configured to collect a set of user data, wherein the set of user data comprises at least one vital health statistic selected from the group consisting of a pulse rate, a body temperature, a respiration rate, and a blood pressure.

An example embodiment 85 including a system for treating a user having PTSD, including: a headset configured to be worn on a head of the user, the headset including a frame, a left light source, a left auditory source, a right light source, and a right auditory source; and a processor communicatively coupled to the headset and a controller, where the processor is configured to receive a sensory stimulation from the controller and transmit the sensory stimulation to the headset, where the sensory stimulation is transmitted at a therapeutically effective amount and the sensory stimulation alternates between: a first sensory stimulation including simultaneously providing a left visual stimulus pattern from the left light source to a left eye of the user and a right auditory stimulus pattern from the right auditory source to a right side of the head of the user; and a second sensory stimulation including simultaneously providing a right visual stimulus pattern from the right light source to a right eye of the user and a left auditory stimulus pattern from the right auditory source to a left side of the head of the user, where the first sensory stimulation and the second sensory stimulation include a first stimulus pattern having a first pulse frequency, a second stimulus pattern having a second pulse frequency less than the first pulse frequency, and a third stimulus pattern having a third pulse frequency less than the second pulse frequency.

An example embodiment 86 including example embodiment 85, where one of the first pulse frequency, the second pulse frequency, or the third pulse frequency is at or between 3.75 Hz and 4.25 Hz, or the first pulse frequency is at or between 7.5 Hz and 8.5 Hz, the second pulse frequency is at or between 2.5 Hz and 3.5 Hz, and the third pulse frequency is at or between 0.5 Hz and 1.5 Hz.

An example embodiment 87 including example embodiment 85, where the processor is programmed to provide the sensory stimulation for a pre-determined treatment period.

An example embodiment 88 including example embodiment 87, where the pre-determined treatment period is approximately 2 hours.

An example embodiment 89 including example embodiment 85, where the first sensory stimulation further includes providing a right tactile stimulus pattern to the right side of the head of the user simultaneously with the left visual stimulus pattern and a right auditory stimulus pattern, and the second sensory stimulation further includes providing a left tactile stimulus pattern to the left side of the head of the user simultaneously with the right visual stimulus pattern and the left auditory stimulus pattern.

An example embodiment 90 including example embodiment 85, where the headset comprises data sensors, wherein the data sensors are configured to collect a set of user data, wherein the set of user comprises at least one vital health statistic selected from the group consisting of a pulse rate, a body temperature, a respiration rate, and a blood pressure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treating a user having post-traumatic stress disorder (PTSD), comprising:
receiving a set of sensory stimulation instructions by a processor in a headset configured for being worn on a head of the user;
responsive to receiving the set of sensory stimulation instructions, administering a therapeutically effective amount of a sensory stimulation to the user, wherein the sensory stimulation alternates between:
a first sensory stimulation including simultaneously providing a left visual stimulus pattern to a left eye of the user and a right auditory stimulus pattern to a right side of the head, and
a second sensory stimulation including simultaneously providing a right visual stimulus pattern to a right eye of the user and a left auditory stimulus pattern to a left side of the head of the user;
wherein the first sensory stimulation and the second sensory stimulation include a first stimulus pattern having a first pulse frequency, a second stimulus pattern having a second pulse frequency, and a third stimulus pattern having a third pulse frequency; and
collecting a set of user data associated with the administering of the therapeutically effective amount of the sensory stimulation to the user.

2. The method of claim 1, wherein one of the first pulse frequency, the second pulse frequency, or the third pulse frequency is at or between 3.75 Hz and 4.25 Hz, or the first pulse frequency is at or between 7.5 Hz and 8.5 Hz, the second pulse frequency is at or between 2.5 Hz and 3.5 Hz, and the third pulse frequency is at or between 0.5 Hz and 1.5 Hz.

3. The method of claim 1, wherein administering the therapeutically effective amount of the sensory stimulation to the user occurs for a pre-determined treatment period.

4. The method of claim 3, wherein the pre-determined treatment period is approximately 5 hours.

5. The method of claim 3, wherein the pre-determined treatment period is approximately 2 hours.

6. The method of claim 1, wherein the first sensory stimulation further includes providing a right tactile stimulus pattern to the right side of the head simultaneously with the left visual stimulus pattern and a right auditory stimulus pattern, and wherein the second sensory stimulation further includes providing a left tactile stimulus pattern to the left side of the head simultaneously with the right visual stimulus pattern and the left auditory stimulus pattern.

7. The method of claim 1, wherein the set of user data comprises at least one vital health statistic selected from the group consisting of a pulse rate, a body temperature, a respiration rate, and a blood pressure.

8. A headset for providing a therapeutically effective amount of a sensory stimulation to a user, comprising:
a frame configured to be worn on a head of the user;
a processor configured to generate a plurality of inputs including a left light source input, a right light source input, a left auditory source input, and a right auditory source input;
a left light source configured to produce, using the left light source input, a left visual stimulus pattern;
a right light source configured to produce, using the right light source input, a right visual stimulus pattern;
a left auditory source configured to produce, using the left auditory source input, a left auditory stimulus pattern; and
a right auditory source configured to produce, using the right auditory source input, a right auditory stimulus pattern,
wherein the processor is further configured to administer, by the headset, the therapeutically effective amount of the sensory stimulation to the user,
wherein the plurality of inputs alternate between:
a first input including simultaneously generating the left light source input and the right auditory source input, and
a second input including simultaneously generating the right light source input and the left auditory source input, and
wherein the first input and the second input include a first stimulus pattern having a first pulse frequency, a second stimulus pattern having a second pulse frequency less than the first pulse frequency, and a third stimulus pattern having a third pulse frequency less than the second pulse frequency.

9. The headset of claim 8, wherein one of the first pulse frequency, the second pulse frequency, or the third pulse frequency is at or between 3.75 Hz and 4.25 Hz, or the first pulse frequency is at or between 7.5 Hz and 8.5 Hz, the second pulse frequency is at or between 2.5 Hz and 3.5 Hz, and the third pulse frequency is at or between 0.5 Hz and 1.5 Hz.

10. The headset of claim 8, wherein the processor is further programmed to provide the sensory stimulation for a pre-determined treatment period.

11. The headset of claim 10, wherein the pre-determined treatment period is approximately 5 hours.

12. The headset of claim 10, wherein the pre-determined treatment period is approximately 2 hours.

13. The headset of claim 8, wherein the plurality of inputs further comprises a left tactile source input and a right tactile source input, and further comprises:
a left tactile source configured to produce, using the left tactile source input, a left tactile stimulus pattern; and
a right tactile source configured to produce, using the right tactile source input, a right tactile stimulus pattern, wherein
the first input further comprises generating the right tactile source input simultaneously with the left light source input and the right auditory source input, and
the second input further comprises generating the left tactile source input simultaneously with the right light source input and the left auditory source input.

14. The headset of claim 8, further comprising data sensors, wherein the data sensors are configured to collect a set of user data, wherein the set of user data comprises at least one vital health statistic selected from the group consisting of a pulse rate, a body temperature, a respiration rate, and a blood pressure.

15. A system for treating a user having post-traumatic stress disorder (PTSD), comprising:
a headset configured to be worn on a head of the user, the headset comprising a frame, a left light source, a left auditory source, a right light source, and a right auditory source; and
a processor communicatively coupled to the headset and a controller, wherein the processor is configured to receive a sensory stimulation from the controller and transmit the sensory stimulation to the headset, wherein the sensory stimulation is transmitted at a therapeutically effective amount and the sensory stimulation alternates between:
a first sensory stimulation including simultaneously providing a left visual stimulus pattern from the left light source to a left eye of the user and a right auditory stimulus pattern from the right auditory source to a right side of the head of the user; and
a second sensory stimulation including simultaneously providing a right visual stimulus pattern from the right light source to a right eye of the user and a left auditory stimulus pattern from the right auditory source to a left side of the head of the user,
wherein the first sensory stimulation and the second sensory stimulation include a first stimulus pattern having a first pulse frequency, a second stimulus pattern having a second pulse frequency less than the first pulse frequency, and a third stimulus pattern having a third pulse frequency less than the second pulse frequency.

16. The system of claim 15, wherein one of the first pulse frequency, the second pulse frequency, or the third pulse frequency is at or between 3.75 Hz and 4.25 Hz, or the first pulse frequency is at or between 7.5 Hz and 8.5 Hz, the second pulse frequency is at or between 2.5 Hz and 3.5 Hz, and the third pulse frequency is at or between 0.5 Hz and 1.5 Hz.

17. The system of claim 15, wherein the processor is programmed to provide the sensory stimulation for a pre-determined treatment period.

18. The system of claim 17, wherein the pre-determined treatment period is approximately 2 hours.

19. The system of claim 15, wherein the first sensory stimulation further includes providing a right tactile stimulus pattern to the right side of the head of the user simultaneously with the left visual stimulus pattern and a right auditory stimulus pattern, and the second sensory stimulation further includes providing a left tactile stimulus pattern to the left side of the head of the user simultaneously with the right visual stimulus pattern and the left auditory stimulus pattern.

20. The system of claim 15, wherein the headset comprises data sensors, wherein the data sensors are configured to collect a set of user data, wherein the set of user data comprises at least one vital health statistic selected from the group consisting of a pulse rate, a body temperature, a respiration rate, and a blood pressure.
